(19) United States
(12) Patent Application Publication
(10) Pub. No.: US 2007/0270667 A1
(43) Pub. Date: Nov. 22, 2007

(54) Musical personal trainer

(76) Inventors: Andreas Coppi, Groton, CT (US); Ronald R. Coifman, North Haven, CT (US); Jonathan Berger, Hamden, CT (US); Frank Geshwind, Madison, CT (US); William G. Fateley, Manhattan, KS (US)

Correspondence Address: FULBRIGHT & JAWORSKI, LLP, 666 FIFTH AVE, NEW YORK, NY, 10103-3198, US

(21) Appl. No.: 11/881,102

(22) Filed: Jul. 25, 2007

Related U.S. Application Data
(63) Continuation of application No. 11/267,080, filed on Nov. 3, 2005, now abandoned.
(60) Provisional application No. 60/624,969, filed on Nov. 3, 2004.
(60) Provisional application No. 60/635,894, filed on Dec. 13, 2004.

(51) Int. Cl. A61B 5/00

(52) U.S. Cl. 600/300

(57) ABSTRACT

Systems and methods for sonification, user influence through sound, and biofeedback, sonification of motion and physiological parameters during physical exercise and the use of music and sound in order to influence the emotional, psychological and physiological state of an exerciser, and the use of sonification and influence in a feedback loop to create a particular exercise experience for a user. A digital music player comprises physiological sensors, a controller, a user interface, a music decoder, a music playback modulator, and an audio data store. The user interface allows a user to specify target values for the physiological sensors as a function of time, the controller selects a playlist of audio data based on the target values, the music decoder decodes the audio data in a sequence corresponding to the playlist, and the music playback modulator causes the sequence and/or the decoding to be modified according to the values of the physiological sensor.

### RELATED APPLICATION

This application claims priority benefit under Title 35 U.S.C. § 119(e) of provisional patent application No. 60/624,969 filed Nov. 3, 2004, and provisional patent application No. 60/635,894 filed Dec. 13, 2004, each of which is incorporated by reference in its entirety.

### FIELD OF INVENTION

The present invention relates generally to systems and methods for sonification, user influence through sound, and biofeedback, and more particularly the present invention relates to systems and methods for sonification of motion and physiological parameters during physical exercise. The systems and methods of the present invention utilize music and sound to influence the emotional, psychological and physiological state of the exerciser, and utilize sonification and influence in a feedback loop to provide a particular exercise experience for the user.

### BACKGROUND OF THE INVENTION

There are several devices in the marketplace for aiding the user to monitor her exercise, such as a chest belt or wristwatch-type monitor. Such monitors are inconvenient in that they require the user to remove some of her clothing each time she wishes to put on the chest belt which contains the heart rate monitor. Also, it is difficult for the person exercising to notice the beep or the display content the wristwatch-type monitor puts out when it receives and processes the signal from the heartbeat sensor in the chest belt.

Wristwatch-type exercise aid devices which detect the pulse wave in the pulse of the person's finger have two shortcomings. The accuracy with which they detect the pulse wave is inadequate, and it is difficult to communicate the appropriate level of exercise to the person while she is exercising.

And no matter whether the person uses an exercise monitor with a chest belt, a wristwatch-type exercise aid device or a treadmill, she is liable to find her exercise routine extremely boring. If the user does not inherently want to exercise, because she does not feel comfortable, and she does not feel inclined to exercise rigorously for fitness, she is unlikely to use the device or system for very long.

We need to find ways to address, however slightly, the societal problem of insufficient exercise. Obesity is increasing at a high rate among both children and adults. It is a contributing cause of both heart disease and cancer. Because “couch potatoes” don't feel like exercising on their own, exercise aid devices must provide enough appeal to get them to want to work out. For people who do not exercise as a routine part of their daily lives, exercise is not enjoyable. Since they do not enjoy it, they do not continue doing it very long. Music has been used for a long time to motivate and energize people while they are exercising. Many people wear headphones and listen to music while exercising. However, not all exercise is good. Too much exercise can be unhealthy.

Appropriate intensity and duration of exercise vary with age, physical strength and level of fitness. No one should exercise if she is sick and is running a temperature. If an elderly person exercises in the same way as a younger person, she may injure her heart, joints or muscles. Furthermore, there are two types of exercise, aerobic and anaerobic. Generally, aerobic exercise is more effective at increasing endurance and reducing body fat, and anaerobic exercise is more effective at increasing muscle strength. The mechanisms which the body uses to generate energy during aerobic and anaerobic exercise are completely different.

Therefore, it is desirable to have a system and method which sonifies the physiological data of the exerciser and plays music in accordance with the physiological data and/or a predetermined goal of the exerciser.

### OBJECT AND SUMMARY OF THE INVENTION

It is an object of the present invention is a system and method for sonification of physiological data. Sonification includes, but is not limited to processes for the communication of one or more parameters (collectively X) to one or more parties. These processes are comprised of the production of one or more sounds, sound patterns, music, tone sequences, and the like (collectively sounds), wherein one or more parameters of the sounds are fixed in value, or varied in time, in some predetermined way, in accordance with the values of X. One of ordinary skill in the art will be familiar with a vast literature relating to sonification.

In accordance with an embodiment of the present invention, physiological sensor data is sonified. The sounds produced can be musical, or can be, for example, one or more simulated environmental sounds such as the sounds of waterfalls, forests, beaches, and other environments. These examples are meant to be illustrative and not limiting, and one of ordinary skill in the art will readily see that there are other possibilities, such as that of so-called white noise or colored noise. In one aspect of the present invention, the sounds are produced in such a way that the auditory cues of synchrony, phase correlation, harmonicity, sensory consonance, musical consonance, rhythmic and metric integration, and other auditory perceptual and cognitive musical attributes are used to create a monitor of the state of the user during physical exercise routines or athletic training and competition. This monitor of state conveys the user's physiological state, and is more easily interpretable than state of the art monitors such as LEDs and video monitors displaying numbers and graphical representations.

In the case of musical sounds, the music can come from pre-recorded music, which would then be modulated in accordance with an embodiment of the present invention, and/or from synthesized music produced in accordance with an embodiment of the present invention.

The physiological sensor data can come from any of a variety of physiological sensors, including but not limited to sensors, as known in the art, that measure: pulse, heart rate, pulse oxygen, blood pressure, temperature, degree of perspiration, walking speed (pedometers), other motions (e.g. a repetition sensor could measure strokes per minute on a rowing machine), breath chemistry (e.g. amounts and ratios of CO, CO₂, and O₂ in the breath, and/or ketones in the breath, etc).

An object of the present invention is to provide a system and method for the influence of the emotional, psychological and physiological state of an exerciser. Sounds (as described herein: musical, environmental, noise, etc) are produced in such a way that the auditory cues (as described herein: synchrony, phase correlation, harmonicity, etc) are. used to create a sound pattern designed to influence the state of the user during physical exercise routines or athletic training and competition. One simple example would be that of using tempo as a means of setting and influencing pace. When it is desired for a runner to take strides at a certain rate, music can be played that has a tempo that matches the desired rate. If an exercise routine is desired where the rate starts at one level, and goes to a next level, and a next, and so on, a device can be programmed in accordance with an embodiment of the present invention, such that the tempo of the music produced or played matches this desired rate as it varies over time.

In accordance with an embodiment of the present invention, the system and method as aforesaid can be combined to produce a feedback loop. A first set of sounds are produced corresponding to a desired physiological state. A second set of sounds are produced, either simultaneously, or sequentially, in order to monitor and convey the user's physiological state. By hearing the differences and similarities, or other comparisons of the structures of the first and second sounds, and optionally the relationship between these sounds and the users own motions and rhythms, the user gets feedback about the difference between his physiological state and the desired state. Alternatively or in addition, a set of sounds can be produced to sonify at any given moment the difference between the user's physiological state and a desired state.

In either case, by making changes to the workout routine, the user can then influence his physiological state, listen to the changes in sound, and bring the physiology in alignment with the desired state. The system of the present invention can then “push” the user into a next desired state in the workout routine, by making gradual or stepwise changes to the desired state, and allowing the user's perception and resonance with the sounds to influence his state.

It is an object of the present invention to provide methods for geometric translation of high dimensional digital data into acoustic perceptual spaces. In accordance with an embodiment of the present invention, regions in a parameter space are translated to sound, such that the auditory perceptual distance between two points corresponds approximately to geometric distance in the parameters. The present invention additionally comprises appropriate dimensional reduction and filtering algorithms and appropriate sound synthesis and processing strategies to effectively elucidate desired sonified features, patterns or attributes in the data.

It should be noted that different embodiments of the invention may incorporate different combinations of the foregoing, and that the invention should not be construed as limited to embodiments that include all of the different elements. Various other objects, advantages and features of the present invention will become readily apparent from the ensuing detailed description, and the novel features will be particularly pointed out in the appended claims.

### BRIEF DESCRIPTION OF THE DRAWINGS

The present invention will be understood and appreciated more fully from the following detailed description, taken in conjunction with the drawings in which:

FIG. 1 shows a block diagram of an embodiment in accordance with the present invention; and

FIG. 2 shows a block diagram of an embodiment in accordance with the present invention.

### DETAILED DESCRIPTION OF THE EMBODIMENTS

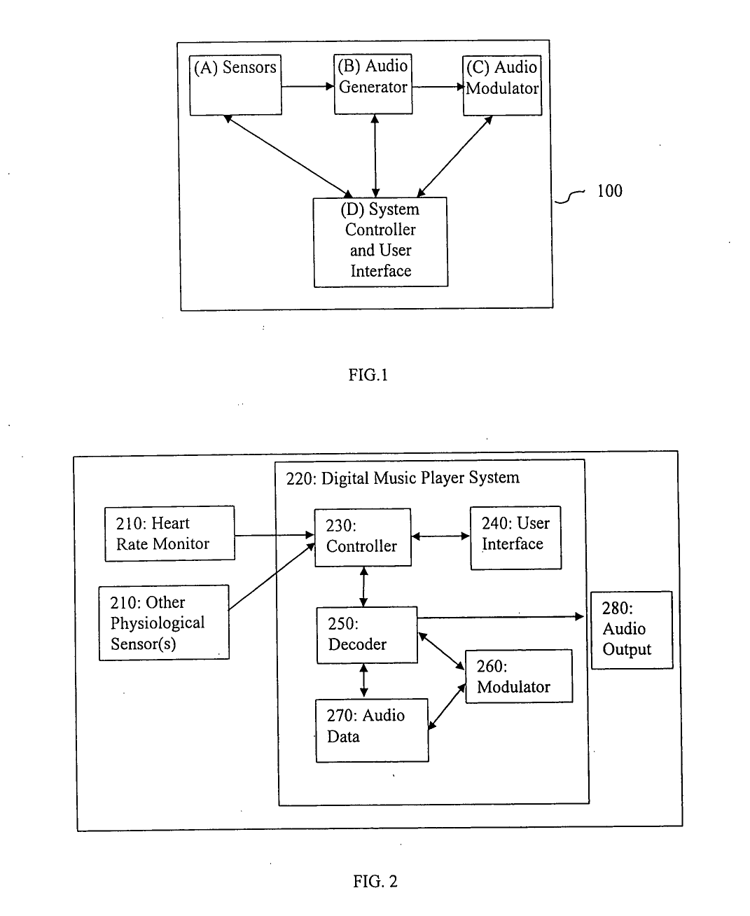

Turning now to FIG. 1, there is illustrated a system/device 100 comprising several co-operating sub-systems, implemented in hardware and software, integrating the functions of physical/physiological/user activity, and real-time parametric audio signal generation and content in accordance with an embodiment of the present invention.

The system 100 comprises sub-systems (A), (B), (C) and (D) which will now be described herein. The sub-system (A) of system 100 comprises one or more sensors for acquiring physiological signals from the system user, including but not limited to sensors, as known in the art, that measure: pulse, heart rate, pulse oxygen, blood pressure, temperature, degree of perspiration, walking speed (pedometers), other motions (e.g. a repetition sensor could measure strokes per minute on a rowing machine), breath chemistry (e.g. amounts and ratios of CO, CO₂, and O₂ in the breath, and/or ketones in the breath, etc).

A second sub-system (B) of the system 100 comprises components that generate audio signals or, specifically, music, including but not limited to, output of synthesized audio, MIDI, or reproduction of stored digital audio samples. Integrated electronic circuits capable of providing all these and more functions are utilized in a range of devices ranging from desktop computers to hand-held communications and electronic game devices, and are known to those of ordinary skill in the art.

A third sub-system (C) of the system 100 comprises components that provide parametric control for the audio generation of the sub-system (B) in accordance with an embodiment of the present invention. For example, the sub-system (C) can control and shape musical and general audio attributes such as tempo, amplitude, timbre, spectral content (equalization), or spatial location within the audio field (balance). In accordance with an embodiment of the present invention, the sub-system (C) can comprise a module for controlling the tempo by varying playback speed according to an evolving pattern of pulses. In accordance with an embodiment of the present invention, the sub-system (C) can comprise a module for controlling the amplitude from total silence to some predefined maximum volume; timbre by spectral addition, subtraction, or frequency modulation; spectral content by equalization; and spatial location by audio channel balancing.

A fourth sub-system (D) of the system 100 comprises components and interface for programming of the interaction of sub-systems (A), (B), and (C) according to a metric or set of metrics quantifying distances within the multi-dimensional parametric space created by sub-systems (B) and (C). In accordance with an embodiment, the sub-system (D) measures distances within the parametric space and utilize those measurements for overall system control. The programming support can be as simple as allowing the selection of one of a number of presets for the various parametric controls exposed in sub-system (C), or as extensive as full programmability support, including logic, driving the controls according to a computer program. A second function of the sub-system (D) is then that of a controller, namely, continuously evaluating the output of sub-system (A), applying rules to that output, translating the result into a stream of parameters and feeding that stream to sub-system (C).

In accordance with an embodiment of the present invention, a sensor in the sub-system (A) comprises a physiological sensor that acquires the user's heartbeat. The sub-system (B) comprises a digital music player, the sub-system (C) comprises a control for the playback rate and the sub-system (D) comprises a user-accessible set of presets allowing the user to choose a desired target heartbeat and musical beat ranges.

FIG. 2 shows a block diagram of a system/device in accordance with an embodiment of the present invention. A series of physiological sensors 210 collect physiological data from a user and send this data to a controller 230 within a digital music player 220. The digital music player 220 additionally comprises a user interface 240, a music decoder 250, a music playback modulator 260, and an audio data store 270.

The interface 240 comprises functionality to allow users to specify how music is to be played over time in accordance an embodiment of the present invention. This can be comprised of the indication of individual songs as well as playlists, target physiological values as a function of time for a workout, and other parameters and options such as how the device should respond when the physiological parameters do or do not meet the target values. In accordance with an embodiment of the present invention, the interface 240 additionally comprises standard digital music player functions such as play, forward, rewind, stop, pause, skip and menu functions as are employed in the art of digital music players.

The controller 230 generally controls the music player 220. In accordance with an embodiment of the present invention, the controller comprises functionality to take and store in memory or otherwise act on information, parameters and commands from the user interface 240. The controller 230 additionally comprises functionality to control the decoder 250 to take music from the audio data store 270, decode it, appropriately modulated by the music playback modulator 260, and send the decoded modulated audio to an audio output 280. The controller 230 additionally comprises functionality to receive physiological data from physiological sensors 210, make decisions about how to modulate the music being played according to the physiological data as well as the information stored in memory from the user interface, and send commands to the music playback modulator 260.

The music playback modulator 260 comprises functionality to adjust the audio signals produced by the decoder 250 in accordance an embodiment of the present invention. This modification comprises of one or more of the following, in accordance with an embodiment of the present invention as disclosed herein: speeding up or slowing down the music in order to match a physiologically and user-interface determined rate at any given time, and/or to reflect the deviation of the physiological rate(s) from the user-interface determined rate, and/or to augment the music with additional sounds to reflect the deviation of the physiological rate(s) from the user-interface determined rate or to reflect a physiologically and user-interface determined rate. In addition to the playback speed of the music, which relates to the tempo of the music, other parameters of the music can be similarly modulated as disclosed herein.

Additionally, the modulator 260 can send signals back to the controller 230, instructing the controller 230 to skip to a different audio track, for example in the case where the desired tempo is very different from the tempo being produced at a given time. Alternatively, the controller 230 can have incorporated functionality to accomplish the same thing.

The audio data store 270 comprises a memory, the contents being comprised of digitally encoded audio segments (described as music herein, but can be other audio as well, such as audio recordings of books, radio or television programs), and can be additionally comprised of other parameters describing these audio segments, such as tempo, pitch, genre, mood and other parameters as used in the art of music and audio characterization and music information retrieval. In accordance with an embodiment of the present invention, the controller 230 as well as the modulator 260 additionally comprise functionality to make decisions and adjustments based on any such additional parameters present in the audio data store 270.

The functionality of storing digital audio data, decoding it, playing it, as well as the functionality of physiological sensing and the electronics and software to implement all such functionality and to control these elements in an integrated way, can be accomplished, for example, by methods know to those of ordinary skill in the art. In order to do so and practice the present invention, such an implementation would additionally be comprised of include the modulation and, sometimes, feedback functions described herein.

Turning now to the detailed explanation of how audio signals are modulated based on measured and target physiological data and other parameters and information, there are several aspects to consider.

In accordance with an embodiment of the present invention, the physiological data and associated parameters are used to construct a mathematical-physics model of a virtual acoustic instrument. For example, such a model can be comprised of a web of masses and springs, or other “material graph” constituting a model of an acoustic instrument. Here, the distances on the graph, and the lengths of the springs, and mass of each of the masses is chosen in a predetermined way based on the characteristics of the physiological data. Each point in a net of points within physiological data space is taken to correspond to one mass or one node in the graph, and the mathematical distances between the points, with distance defined in a predetermined way, is used in a predetermined way to set the distance between the masses, and/or the lengths and spring constants in a mass and spring model.

In this aspect, by virtually “striking” a given data point x on the virtual instrument, the instrument is set into vibration in a unique way.

More specifically we can view eigenfunctions of an operator on the data set as coordinates, φᵢ(x), corresponding to eigenvalues λᵢ, and the corresponding sound at data point x would be Σφᵢ(x)cos(λᵢt). Such coordinates tend to be naturally supported on different clusters, and would result in different sound for data points in different clusters. Like the interaction of natural resonances of a musical instrument with the place and method, these resonances are energized and these clusters would result in different sound patterns.

Appropriate subsets of eigenfunctions can be used to parametrize the data in low dimensions for both visualization and sonification. Of course the formula given above is but the simplest of illustrations, and is not meant to be limiting. Indeed, modifications will suggest themselves to those of skill in the art, modifications which make the system more perceptually effective and metrically accurate. In particular, the models suggested in U.S. patent application Ser. 11/165,633, filed Jun. 23, 2005, which is incorporated herein by reference in its entirety, can be used in embodiments of the present invention. The diffusion maps constructed there provide a translation from complex data into a small set of numbers so that the conventional Euclidean metric represents a meaningful inference on the data. The sonification is designed so that the perceptual distance between sound streams relates directly to the Euclidean distance in parameter space.

In accordance with an embodiment of the present invention, two examples of sonification methodologies include: 1) sonification of data by creating vowel-like sounds using filters and mapping dimensions to the center frequency and bandwidth settings of filters anchored around a typical vowel sound, and 2) mapping dimensions to onset and duration.

There are two general approaches to sonifying data, which we term Parameter Mapping and Metaphoric Modeling. In the parameter mapping approach, numeric values from a data set are mapped to sound synthesis attributes such as frequency, amplitude, modulation index, etc. Metaphoric modeling sets data states to well known auditory metaphors (e.g. vowel sounds) to provide an intuitive sonification in which sought after events are readily recognized.

As an example, both methods can be employed on a set of physiological sensor data as described herein. Applying the parameter mapping approach, first scaled data is mapped to various parameters of a complex tone in which an individual partial is associated with a particular data dimension. Several implementations are possible, including but not limited to:
1. Mapping data to the amplitudes of each partial of a complex harmonic tone with a set fundamental frequency to produce timbral variations of a harmonic tone.
2. Mapping data to the frequencies of individual partials to create inharmonic spectra in which the timbral quality and degree of inharmonicity represents particular data states.
3. Mapping subsets of the data tuned to components of musical triads.
4. Mapping data to temporal offsets in order to create melodic sequences rather than harmonic events.

In an embodiment incorporating the metaphoric modeling approach, a filter bank used with formant like resonance peaks to create vowel like sounds. In various embodiments data can be mapped to center frequencies, bandwidths and/or amplitudes of the formants. Alternately, the data can be anchored to particular vowel sounds to produce a situation in which a particular state of the data is mapped to a particular vowel, and the percept of relative proximity to that vowel attained meaning.

In accordance with an embodiment of the present invention, the system and method adapts waveguide models. Digital waveguide models are discrete-time models of distributed media such as vibrating strings, bores, horns or plates. They are often combined with models of lumped elements such as masses and springs. There are efficient digital waveguide models of string, brass and wind instruments and data mappings can be created to drive excitations of these models.

In accordance with an embodiment of the present invention, one can incorporate computational models for auditory cortex processing (e.g. Shihab Shamma, On the role of space and time in auditory processing, TRENDS in Cognitive Sciences Vol. 5 No. 8, August 2001) to enable accurate translation of regions in data parameter space to auditory cortex (AC) parameters, effectively creating Auditory Perception Models to fit the data. The precise understanding and emulation of the cochlea to auditory cortex map is critical for faithful conversion of geometry to perception. (See also K. Wang and S. Shamma, Wavelet Representations of Sound in the Primary Auditory Cortex, J. Optical Engineering, 33(7), pp. 2143-2148, 1994; K. Wang and S. Shamma, Representation of Acoustic Signals in the Primary Auditory Cortex, IEEE Trans. Audio and Speech Processing, V3(5), pp. 382-395, 1995).

In accordance with an embodiment of the present invention, a Musical Personal Trainer involves the sonification of motion and bodily functions during physical exercise in order to monitor a specialized assessment of performance. Various motion and physiological sensors are employed, and their data integrated, in order to create an auditory scene, whether musical or environmental, in which the auditory cues of synchrony, phase correlation, harmonicity, sensory consonance, musical consonance, rhythmic and metric integration, and other auditory perceptual and cognitive musical attributes are used to create an easily interpretable monitor of the state of the user during physical exercise routines or athletic training and competition. A Musical Personal Trainer relies upon the many exercise routines performed while listening to music and the desire for exercisers to have access to real-time response and a signal when they are in or out of the ‘comfort zone’, a predetermined state in which the individual is optimally achieving the desired benefits of the exercise.

As an example of such an embodiment, consider a jogger fitted with basic sensors connected to a small portable sonification device, perhaps integrated into a digital music player, or cellular telephone with MIDI, polyphonic FM, or digital music capability. The user sets a target pace—which sets a basic metrical pulse or drumbeat. In the simplest case, the runner knows when that pace is met when the sonified gait matches the target drumbeat. Other monitors can be mapped to particular musical characteristics (timbral, musical or both) and the degree of perceived correlation between these represents the degree to which the user is in the routine's ‘comfort zone’ of the exercise. Deviation from this zone from any sensor parameter can easily be heard both in terms of the nature and degree of the musical or auditory deviation.

In an embodiment of the present invention, the employed sonification schemes are comprised of a range of preset auditory mappings such as but not limited to:
a. A mode in which sensor rate and regularity is mapped to sample rate such that the playback speed of any digital audio file can be controlled by the runner's pace, while the digital EQ, filtering, and effects can be controlled by heart rate sensors, etc.
b. A mode in which heart rate target is mapped to a predetermined musical motif, and other sensors to contrapunctal motives that emerge when the exercise routine is in the ‘comfort zone’.

Such an embodiment of the present invention comprised a game in which a unique musical composition is created during each exercise routine.

As a further illustration of such embodiment, a user maps a unique drum sound, pattern and/or beat position to each sensor. User defined or selected musical or auditory mappings are made such that an ideal ‘target musical piece’ that represents a healthy and optimal workout session can be generated. During exercise a new composition is created based on the sensor feedback. This and any subsequent workout can be recorded. Archived recordings can be compared to chart improvement. An embodiment of the present invention additionally comprises components to upload these and other data to a website, and a website social network for the sharing of these data and other social interaction.

These and other sonification schemes can be implemented using technology including but not limited to devices that implement standard MIDI and digital audio formats and methods such that auditory realization can be integrated into the devices. Such standard devices include but are not limited to cellular phones, PDAs (Portable Digital Assistants), and digital music players.

In order to implement many of the sonification methodologies described herein, several computer music methods known to those skilled in the art can be used. For signal processing and alteration of existing pre-recorded digital or MIDI-encoded music, standard methods can be used to effect sample rate change and digital filtering and effects such as delay, bandpass, etc. MIDI representations of any existing or original musical composition can be used to make high-level musical alterations in real-time. Beat tracking algorithms (see for example, Scheirer, Eric D., Tempo and Beat Analysis of Acoustic Musical Signals, J. Acoust. Soc. Am. 103:1, January 1998, pp 588-601) can lock into the underlying metric pattern of a pre-recorded piece for synchronization or alteration.

One embodiment of a real time musical adaptation method is comprised of employing a predetermined number of sets of ‘skeletal’ reductive representations of music to provide a multi-track framework upon which surface level ‘patterns’ can be placed. The ‘guide tones’ in a given track and the harmonic summary of the locale within the skeleton dictate how the music is to be adapted to ‘fit’ harmonically, rhythmically, metrically, etc.

The Musical Personal Trainer can also implement an interface or include a remote software package allowing the user to design a playlist of music to accompany a workout routine. Musical selections can be made based on musical parameters including but not limited to tempo, genre, percussivity, etc. which serve to motivate and optimize the desired pace, strain, duration, and effectiveness of the particular exercise at that point in the routine.

In accordance with an embodiment of the present invention, the system utilizes feedback to interact with the user. The exercise routine customized playlist as disclosed herein can be further augmented with alternate musical selections for each of the exercise segments. These alternate selections are chosen to motivate either an increase or decrease in expended effort. During the course of the exercise routine, the system monitors physiological sensor data and, based on whether the user's performance is exceeding or falling short of the pre-defined desired regime for that exercise segment, the system can make a decision to change the musical selection to one of the alternates. This is an example of a feedback loop where the device uses sensor input and musical output to affect the user's actions. Alternately, the reverse is possible. The user's action can be used to drive the musical or sonified output of the device.

For example, a user may wish to set the pace of an exercise him/herself during the exercise routine based on comfort, mood, energy, and other factors. In this case, the user's behavior can be quantified with motion and physiological sensor data from which the system will infer, in a predetermined way, a new definition of musical parameters and react accordingly, e.g. change tempo, musical selection, etc. In another example, the physiological sensor data may indicate an increasingly unhealthy or even dangerous state of the user, and react by slowing the tempo or modifying the sonified feedback in a way to alter the user's exercise pace and or effort in order to better fit the user's apparent performance despite a previously defined regimen. In both of these cases, the user's behavior is intentionally driving the music and or sonification. It is also the case that similar feedback loops can be constructed that allow for both passive and active interaction with the user.

As discussed herein, altering the tempo of music is one way to affect the pace of the user during an exercise. The change in tempo can be accomplished with aforementioned known techniques that increase or decrease tempo while sufficiently preserving the original pitch and quality of the music or sonification. Alternately, a simpler approach is to have a library of musical selections, rhythms, or other sonified passages that span the desired range of tempos. The system can choose an appropriate selection from this library to match the desired tempo. In the case where an exact tempo is not available, the two approaches can be fused, and the processing approach can be used to alter the tempo of the closest available match from the library.

In all of the described sonification methodologies, musical ‘coherence’ at whatever level can be an auditory target in auditory feedback based sonification. Using the same technology, a wide range of applications include but are not limited to GPS-based in-car traffic flow sonification, athletic performance improvement methods and biofeedback relaxation.

One such example is that of a sleep aid, dubbed the “Composure Composer.” Biofeedback sensors comprised of one or more of respiration, heart rate and blood volume pulse, electrodermal response, skin temperature, and electrical activity of specific muscles, are mapped to auditory displays that infer the degree of correlation, particularly in terms of musical harmoniousness (in the general musical sense of ‘sounding good together’). The auditory feedback can be used both as a monitor and as a means of setting and meeting a particular goal. The goals can be adapted for promoting relaxation or sleep.

A similar device can be used for remote baby sleep-monitoring and automatically generating sleep-inducing music and or rhythms that respond to infant biofeedback through crib-side speakers.

In many of the embodiments discussed herein, the sonification methodologies can be superimposed onto an audio track of complimentary or non-musical nature that the individual desires to listen to during the exercise routine. In this way the desired biofeedback and performance enhancement can take place while the individual is simultaneously listening to other multimedia content, live or prerecorded, such as but not limited to news reports, narrated books and print media, radio and internet audio streams, video, or television programs.

It should be noted that different embodiments of the invention may incorporate different combinations of the foregoing elements and aspects of the invention, and that the invention should not be construed as limited to embodiments that include all of the different aspects.

It is to be understood that the described examples and embodiments are merely illustrative of some of the many specific embodiments that represent applications of the principles of the present invention. As those of ordinary skill in the art will appreciate, numerous and varied other arrangements may be readily devised without departing from the scope of the invention.

While the foregoing has described and illustrated aspects of various embodiments of the present invention, those skilled in the art will recognize that alternative components and techniques, and/or combinations and permutations of the described components and techniques, can be substituted for, or added to, the embodiments described herein. It is intended, therefore, that the present invention not be defined by the specific embodiments described herein, but rather by the appended claims, which are intended to be construed in accordance with the well-settled principles of claim construction, including that: each claim should be given its broadest reasonable interpretation consistent with the specification; limitations should not be read from the specification or drawings into the claims; words in a claim should be given their plain, ordinary, and generic meaning, unless it is readily apparent from the specification that an unusual meaning was intended; an absence of the specific words “means for” connotes applicants' intent not to invoke 35 U.S.C. §112 (6) in construing the limitation; where the phrase “means for” precedes a data processing or manipulation “function,” it is intended that the resulting means-plus-function element be construed to cover any, and all, computer implementation(s) of the recited “function”; a claim that contains more than one computer-implemented means-plus-function element should not be construed to require that each means-plus-function element must be a structurally distinct entity (such as a particular piece of hardware or block of code); rather, such claim should be construed merely to require that the overall combination of hardware/firmware/software which implements the invention must, as a whole, implement at least the function(s) called for by the claim's means-plus-function element(s).

It is to be understood that the described examples and embodiments are merely illustrative of some of the many specific embodiments that represent applications of the principles of the present invention. As those of ordinary skill in the art will appreciate, numerous and varied other arrangements may be readily devised without departing from the scope of the invention.

1-2. (canceled)

3. A method of sonifying affinity between points of a set of digital data, comprising the steps of:
defining a relevance or affinity metric and associated affinity graph on the points of said set of digital data;
computing associated diffusion mapping and applying said associated diffusion mapping to embed said set of digital data into a low-dimensional Euclidean space such that a geometric distance between mapped points of said set of digital data is substantially similar to a diffusion distance between unmapped points of said set of digital data; and
associating a synthetic sounds to said mapped points of said set of digital data so that a perceptual auditory distance between said mapped points of said set of digital data corresponds to said geometric distance between said mapped points of said set of digital data.

4. The method of claim 3, wherein the step of associating comprises the step of associating at least timbre of said synthetic sound to said mapped points of said set of digital data.

5. The method of claim 3, further comprising the step of sonifying digital physiological states of a person which comprises the steps of:
measuring time-series readings of a plurality physiological sensors comprising at least one of the following: heart rate monitor, blood pressure sensor, pulse-ox sensor, temperature sensor, or accelerometer; and
organizing said time series readings into said set of digital data and defining a notion of affinity or similarity among said time-series readings.

6. An interactive digital musical synthesizer enabling biofeedback, comprising:
a physiological sensor or array of physiological sensors;
a controller;
a user interface for allowing a user to specify target auditory responses for physiological sensor parameters;
a music decoder or player; and
a music synthesizer for generating sound to be played by said music decoder or player by sonifying digital physiological states of a user by:
measuring time-series readings of a plurality physiological sensors comprising at least one of the following: heart rate monitor, blood pressure sensor, pulse-ox sensor, temperature sensor, or accelerometer;
organizing said time series readings into said set of digital data and defining a notion of affinity or similarity among said time-series readings;
defining a relevance or affinity metric and associated affinity graph on the points of said set of digital data;
computing associated diffusion mapping and applying said associated diffusion mapping to embed said set of digital data into a low-dimensional Euclidean space such that a geometric distance between mapped points of said set of digital data is substantially similar to a diffusion distance between unmapped points of said set of digital data; and
associating said sound to said mapped points of said set of digital data so that a perceptual auditory distance between said mapped points of said set of digital data corresponds to said geometric distance between said mapped points of said set of digital data; and
wherein said user responds to said sound, thereby affecting said physiological sensor parameters of said user in a biofeedback loop.

7. A method enabling biofeedback while a user continues to listen to a selected audio content, comprising the steps of:
selecting an audio stream from multimedia content by said user, said multimedia content comprises at least one of the following: music, news reports, narrated books, print media, radio and Internet audio streams, video, and television programs;
deriving a synthesized audio stream by sonifying physiological sensors and part of a biofeedback loop by:
measuring time-series readings of a plurality physiological sensors comprising at least one of the following: heart rate monitor, blood pressure sensor, pulse-ox sensor, temperature sensor, or accelerometer;
organizing said time series readings into said set of digital data and defining a notion of affinity or similarity among said time-series readings;
defining a relevance or affinity metric and associated affinity graph on the points of said set of digital data;
computing associated diffusion mapping and applying said associated diffusion mapping to embed said set of digital data into a low-dimensional Euclidean space such that a geometric distance between mapped points of said set of digital data is substantially similar to a diffusion distance between unmapped points of said set of digital data; and
associating said synthesized audio stream to said mapped points of said set of digital data so that a perceptual auditory distance between said mapped points of said set of digital data corresponds to said geometric distance between said mapped points of said set of digital data; and
wherein said user responds to said synthesized audio stream, thereby affecting said physiological sensor parameters of said user in a biofeedback loop; and
superimposing said synthesized audio stream onto said user-selected audio stream at a level selected by said user such that said user's perception and comprehension of both streams are not affected.
